(19) United States
(12) Patent Application Publication
(10) Pub. No.: US 2023/0360786 A1
(43) Pub. Date: Nov. 9, 2023

(54) SYSTEM AND METHOD FOR PROVIDING A SURGICAL WORKFLOW TOOL

(71) Applicant: Joseph Luna, Stafford, VA (US)

(72) Inventor: Joseph Luna, Stafford, VA (US)

(21) Appl. No.: 18/084,340

(22) Filed: Dec. 19, 2022

Related U.S. Application Data
(60) Provisional application No. 63/256,935, filed on Oct. 18, 2021.

(51) Int. Cl. G16H 40/40; G16H 40/20; G16H 40/67; G16H 70/20; G16H 80/00

(52) U.S. Cl. CPC G16H 40/40; G16H 40/20; G16H 40/67; G16H 70/20; G16H 80/00

(57) ABSTRACT

A system for providing a surgical workflow tool is disclosed, including at least one user computing device in operable connection with a user network. An application server in operable communication with the user network to host an application system for providing a surgical workflow tool. The application system having a user interface module for providing access to the application system through the user computing device. The application program assists in supply chain maintenance, scheduling, and hardware integration of the surgical process of a healthcare facility.

### CROSS REFERENCE TO RELATED APPLICATIONS

The present application claims priority to U.S. Provisional Patent Application No. 63/256,935 filed Oct. 18, 2021, entitled “SYSTEM AND METHOD FOR PROVIDING A SURGICAL WORKFLOW TOOL” which is hereby incorporated by reference in its entirety.

### TECHNICAL FIELD

The embodiments generally relate to systems and methods for providing workflow tool for the healthcare field and more specifically to a system and method for providing a surgical workflow tool.

### BACKGROUND

Surgical preference cards contain a list of instruments, equipment, and supplies that a surgeon needs to efficiently perform a particular procedure. In such, the preference card allows the surgeon to customize their needs for each procedure and ensure that they have the correct supplies available. Current systems fail to provide supply cost tracking, data collection mechanisms, real-time clinical communications, access to specific media and do not allow the management of team members in a portable platform. Further, hardcopy preference cards cannot be edited, leading to outdated information and general inefficiencies in the process.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments provided herein relate to a systems and methods for providing a surgical workflow tool including at least one user computing device in operable connection with a user network. An application server in operable communication with the user network to host an application system for providing a surgical workflow tool. The application system having a user interface module for providing access to the application system through the user computing device. The application program assists in supply chain maintenance, scheduling, and hardware integration of the surgical process of a healthcare facility.

Facilities generally write staff assignments the preceding evening, giving the staff little to no indication of the surgeries they will be expected to assist in the next day. Rather than finding out staff assignments that morning, the embodiments will alert staff members on their cell phones the second the assignments are created. This feature connects the surgeon with his/her staff.

Supply chains are in operating rooms are bare, containing nothing but serial numbers and text presented in a disorienting fashion. The embodiments provide the ability to post images and videos on any supply to assist the user in properly using and caring for that supply. Especially in a large operating room setting, there is no streamline way for staff members to locate supplies they are not already familiar with. The system provides images and descriptions of all supply locations to point the user to the item's location in the quickest possible manor.

Informatics for ASCs and especially large hospitals are generally a mess. Inaccurate information manually entered by a circulating nurse (who is rushing) leads to inaccurate supply usage reporting as well as inaccurate procedure times, making data analysis quite a challenge. By creating a manual, accurate, and comprehensive data recording system, circulating nurses and other staff members are able to utilize text free, chronological data inputs to accurately record procedure data while requiring minimal time and effort, allowing for better patient care.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a computing system, according to some embodiments; and

FIG. 2 illustrates a block diagram of a computing system and an application program, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

In general, the system is a is a surgical workflow tool that increases the efficiency and effectiveness in which clinicians, administrators, and all perioperative staff are able to perform their jobs. The systems and methods replace the preference card system with a completely digitized, mobile surgeon preference database that includes pictures, videos, and a social area where peers can share their knowledge of a particular procedure. Preference cards are notoriously difficult to create and properly maintain, the embodiments provide easy, comprehensive pathways to easily and efficiently edit procedure preferences. Preference cards are historically only accessible by a few individuals, the embodiments allow both administrators and surgeons to designate surrogate users to maintain surgical preferences on their behalf.

In some embodiments, the system improves perioperative communication with a mobile, HIPAA compliant messaging and alert platform that creates surgical staff communication groups for each procedure to encourage preoperative communication and enhance staff preparedness. Current methods for staff communication involve cell phones, overhead paging systems, and pagers. The system allows staff to use familiar pathways of communication thereby enhancing communication efficiency. Procedure milestone alerts perfectly coordinate the surgical staff, anesthesia, xray, and the surgeon with live time procedure updates.

Economic potential and commercial applications may include using voice recognition software to be used as a virtual assistant in the operating room.

Hospitals, ASCs, and medical clinics of all types may utilize the embodiments provided herein to increase various metrics associated with their practice, including annual patient volume, patient care and satisfaction, as well as staff participation and job satisfaction.

By utilizing the system in the supply chain department, facilities/practices can expect to see a dramatic decrease in supply waste and supply ordering mistakes with the new founded ability to easily edit supply fields, educate staff and practitioners on proper supply use, and accurately track supply usage will receiving relevant alerts regarding supply inventory and supply expiration.

With a volume based model, the embodiments will capitalize on heavy user traffic by offering non-obvious (subliminal) advertisement to supply vendors at a high premium. Vast hardware capabilities and potential applications allow for capitalization on mobile device sales such as but not limited to: Tablets, television monitors, desktop monitors, cell phones, smart watches, smart glasses, speakers, cameras, and charging stations. This is achieved while users engage in daily clinical and administrative workflow. Users will incorporate platform features to address clinical and administrative pain points. The core features of the system enable users to utilize sub features of each main function as described below.

In some embodiments, the system provides a proprietary process of creating new clinical and administrative workflows by all users which yields data and or commerce opportunities allow for 3D printing clinical supplies and instruments. This is interfaced in the supply function. Augmented reality, 3D printing, data collection, data reporting, data analysis, cost collection, cost reporting, cost analysis, communication tools, Social Media channels, Safety protocol templates, management of staff schedules, management of the surgical schedule, delivery of patient information, are some examples of sub core features.

Potential opportunities to scale the platform will be developed as it relates to commerce, marketing and advertising derived from new clinical and administrative workflow of users incorporating platform features administering patient care and integrating this entire process in a modular structure. This process is delivered using the adjunct of the functions of the system's platform.

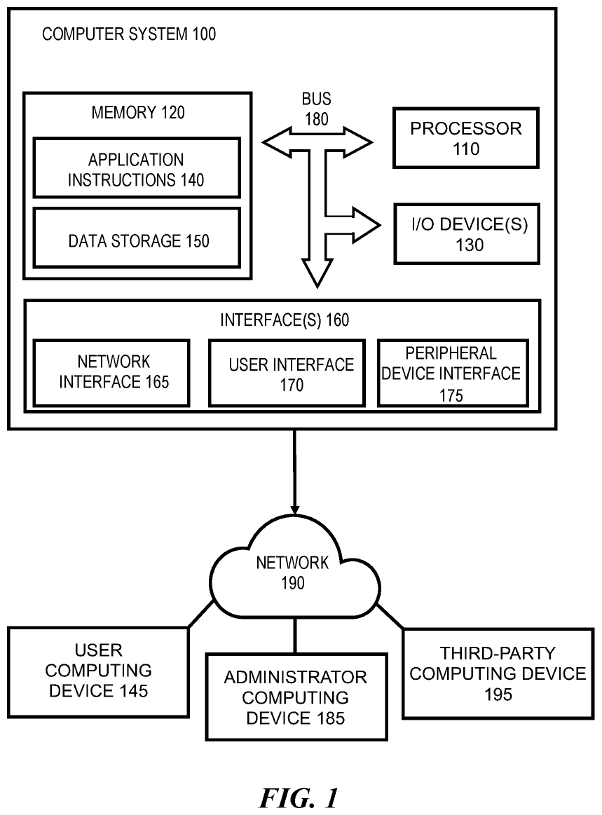

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 140, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The user computing device 145 may be utilized to establish credentials, create a user profile, and interact with the surgical workflow system. The third-party computing device 195 may be utilized by third parties (e.g., auxiliary staff and others in communication with the surgical workflow system) to receive communications from the user computing device and/or administrative computing device 185.

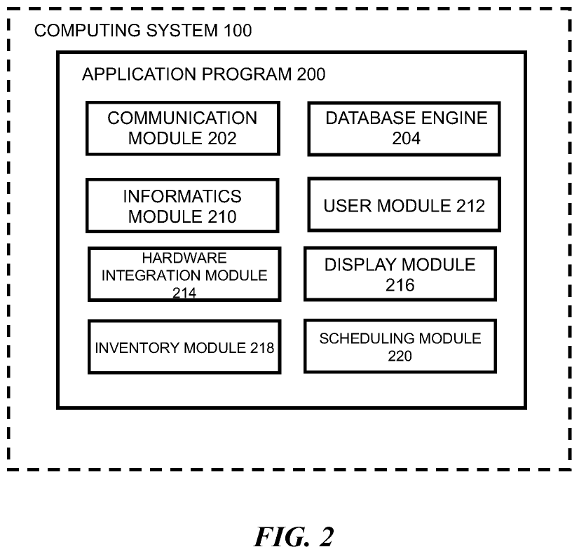

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, an informatics module 210, a user module 212, a hardware integration module 214, a display module 216, an inventory module 218, and a scheduling module 220.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195. In some embodiments, the communication module 202 allows each user to transmit and receive information which may be used by the system.

The communication module 202 may be capable of generating digital preference cards utilized by the user. In some embodiments, the communication module 202 may include a social media message board which is separated by service lines and localities. A messaging platform may be operated by the communication module 202 to enhance operating room communication using HIPPA compliant messaging (e.g., secure text messaging, video messaging, etc.) which may use wireless communication networks and be delivered to hardware components (e.g., augmented reality glasses) and/or smart devices.

The communications module 202 may also enhance operating room communication using the messaging feature by increasing staff knowledge and preparedness through delivery of information relating to supply, instruments, and equipment. Critical and comparative statistical analysis and reporting may be transmitted to relevant users such that efficiency is optimized while reducing the potential for waste (such as in hardware or personnel resources).

In some embodiments, the communication module 202 may facilitate HIPAA compliant cell phone messaging between staff, surgeons, administrators, third-parties, and all user roles available within the platform. Group conversations may be created in unison with the calendar function for each specific procedure.

In some embodiments, the communication module 202 may generate case milestone alerts which can be transmitted to relevant users. The case milestone alerts may be transmitted once particular (predetermined) stages of patient care are reached.

In some embodiments, the communication module 202 permits users to post images, video files, text, and share YouTube videos to help keep relevant peer users up to date on new techniques and equipment being introduced into that specific service line. Users can pick which service forums they would like to subscribe to and can elect to receive alerts from that service line. Specialty coordinators can make announcements to all users that have a specific service chosen as a specialty. In some embodiments, a global forum is available as well that connects all the system's clients in one place where that specific service line can be discussed.

In some embodiments, the communication module 202 may enable video chat sessions to allow surgeons to consult with outside sources (i.e., third-parties) intraoperatively while providing the outside source with visual display of the surgical environment.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. The database engine 204 allows each user and module associated with the system to transmit and receive information stored in various databases.

In some embodiments, the informatics module 210 allows for data collection and cost analysis of various aspects of the user's interactions with the system and the clinical site. For example, the user will use the system to perform daily tasks which yield data to the system which is collected and reported in the system application. Users will use the platform to select instruments, supplies, and equipment needed to perform surgical procedures. Utilization data of these items for individual procedures will be reported and managed within a cost analysis and revenue cycle component of the system, operated by the informatics module 210.

In some embodiments, the informatics module 210 is capable of communicating with 3-Dimensional (3D) printing systems to enable users to 3D print instruments, supplies, and equipment (which may be collectively referred to herein as “hardware”).

In some embodiments, the informatics module 210 is capable of communicating with augmented reality systems to enable users to perform various functions. This may include communicating with other users, or otherwise interacting with the system. In one example, the informatics module 210 may display informatics data collected by the system.

In some embodiments, the informatics module 210 provides text-free procedural statistics tracking abilities which are presented to the user. The procedural statistics lessen data recording errors and present the procedural statistics in a comprehensible, chronological fashion. Perioperative procedural data is collected, stored, reviewed, and compared as it relates to a patient, patient demographic, staff member, staff, demographic, surgeon, surgeon demographic, or the facility as a whole or as it compares to other surgical facilities. Detailed patient demographics allow for patient diagnosis, allergies, previous surgeries, health risks, scans, and a variety of other patient characteristics to be factored into the analytical process. The system applies supply cost, staff salaries and a variety of other expenditures to provide the most accurate procedure cost analysis possible.

Reportable data is produced by populating the systems algorithms with one or more of the following patient-specific milestones, including but not limited to: Patient Arrival, Patient in Pre-Op, Room Opened, Patient in Room, Patient Asleep, Foley Inserted, Patient Prepped, Incision Made, Closing Started, Dressing On, Patient Awake, Patient Exits Room, Patient in Recovery, Patient Discharged, Count performed, and Time-Out performed.

### System Algorithms

(a) Patient wait time
   (i) (Patient in Pre-Op)−(Patient Arrival)
(b) Pre-op time
   (i) (Patient in Room)−(Patient in Pre-Op)
(c) OR setup time
   (i) (Patient in Room)−(Room Opened)
(d) Patient asleep time
   (i) (Patient Asleep)−(Patient in Room)
(e) Positioning time
   (i) (Patient Prepped)−(Patient Asleep)
(f) Procedure prep time
   (i) (Incision Made)−(Room Opened)
(g) Procedure time
   (i) (Dressing On)−(Incision Made)
(h) Closing time
   (i) (Dressing On)−(Closing Started)
   (i) Patient awake time
   (i) (Patient Awake)−(Dressing On)
(j) Patient recovery time
   (i) (Patient Discharged)−(Patient in Recovery)
(k) Total patients stay
   (i) (Patient Discharged)−(Patient Arrival)
(l) OR productive time
   (i) (Patient Exits Room)−(Patient in Room)
(m) OR non-productive
   (i) ((Patient in Room)−(Room Opened))+(OR turnover)
(n) OR turnover time
   (i) (Patient in Room #2)−(Patient Exits Room #1)
(o) On-Time start
   (i) (Patient in Room)−(Scheduled Start Time)

Reportable data is produced by populating the systems algorithms with one or more of the following patient-specific milestones, including but not limited to: Patient Arrival, Patient in Pre-Op, Room Opened, Patient in Room, Patient Asleep, Foley Inserted, Patient Prepped, Incision Made, Closing Started, Dressing On, Patient Awake, Patient Exits Room, Patient in Recovery, Patient Discharged, Count performed, and Time-Out performed.

Individual user data tracking is produced from procedure data and includes individual users that have been assigned to the procedure. As users complete more cases, user performance data is collected and delivered in a comparative (avg) fashion.

Procedure supply usage includes surgeon-specific procedure supply preferences which are predetermined by the “open” and “hold” quantities. Circulating nurses confirm the supplies that are utilized in the specific procedure. The circulator will have the ability to add items to the supply list that are not pre-populated via preference card. A combination of individual supply cost and circulator provided “quantity used” values are used to extrapolate the specific supply cost or that procedure.

Facility statistics tracking includes collected procedure data can be manipulated to show year-t-date averages as well as monthly averages. Data can also be manipulated to compare individual users within a specific role.

Patient data can play factors in treatment times. The following patient data factors are recorded: Date of birth, Gender, Height, Weight, Pre-op diagnosis, post-op diagnosis, Allergies, Previous Surgeries, and Supply Chain.

In some embodiments, the user module 212 facilitates the creation of a user account for the application system. The user module 212 may allow the user to input account information, common procedure information, preferences and the like.

In some embodiments, the user module 212 may permit the user to input procedure preferences. Surgeons and administrators assign surrogate users the ability maintain procedure preferences on their behalf (by surgeon). Users can post videos and pictures specific to that surgeon's procedure.

In some embodiments, supply and equipment preference data can be updated instantly by the surgeon, an administrator, or any user with surrogate privilege for that surgeon. Supply and equipment preferences may be input via the user and stored in the database as facilitated by the user module 212. Working in tandem with the supply chain data collection, the communication module 202 alerts relevant parties when a supply has been placed on backorder, gone out of stock, or has simply been replaced, and offers to switch to a comparable product. Supply and equipment changes are applied across all procedure preferences with one click.

In some embodiments, the system provides inventory data that alerts relevant parties when products are about to expire and require prompt use to avoid waste. Supply “Replacement” functions allow for all procedure preferences to be updated instantaneously as a supply is replaced on the shelf for any various reason. This process is facilitated by the user module 212.

In some embodiments, a location database allows the facility to create and manage various supply and equipment locations and provide location details and images. By populating these locations with supplies users can always expect a streamline path to supply acquirement. Images and/or video posts are available for every supply, providing a channel for proper usage instructions.

In some embodiments, “Vendor” supply data assignment links the supply vendor of that particular product to a “Vendor” user that is assigned to that facility. These “Vendor” users can be granted the ability to edit all supplies created by their company which can limit some staff burden.

In some embodiments, a supply cost field is utilized in coalition with procedure data collection to provide accurate procedure cost reports.

In some embodiments, the hardware integration module 214 allows for various hardware components to be integrated with the system. This integration allows for data to be transmitted and/or received from the hardware. As used herein, hardware may include smart devices, tablets, computers, smart watches, smart glasses, wearables, 3D printers, and the like.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored.

The applications program includes an application program interface (API) to integrate the various functionalities described herein. The system will initially seek API functionality and user module 212 to assist in supply chain setup is facilitated by the API to avoid data entry replication and to limit software integration costs. The system will work the facility's current software to ensure every supply is transitioned to a facility account. From there additional supply data offered by the system can be populated.

Procedure preferences can be input via the API and user module 212. Working off of the supply chain, user, and procedure database, an API will be utilized to create initial instances of surgeon-specific procedures as well as initially populate the procedure with supply preferences.

Patient Imaging can be performed using the API. When a patient is booked and the “MRN” field is provided, the system will pull all available imaging on that specific patient and place it in the procedure dashboard.

In some embodiments, during an “account build” procedure all supplies, will be added to a client supply database. Further, procedure preferences will be added to client supply database. During an “account maintenance” procedure, new supplies will be added to the client supply database. However, if that supply is already present in client supply database, edit actions will never be pre-authorized and this event would require human intervention. Procedure preferences edit actions are be performed with a manually called sync function.

In some embodiments the system includes an inventory module 218 which may be configured to store and allow the user to view supply, instrument, and equipment catalogs which may include digital count sheets. The inventory module 218 par levels and expiration dates may be transmitted to the user via notifications.

In some embodiments, the system includes a scheduling module 220 to operate and manipulate a calendar/scheduling system. User schedules may be updated as work and case assignments are created. Procedure booking allows a booking user to pick available case times that will not conflict with future or undergoing procedures. In some embodiments, the procedure booking, and schedule change alerts are instantaneously sent to relevant users via the communication module 202. Administrative users can easily assign staff members to a particular case or operating room. Each scheduling update is transmitted to the system's informatics platform.

The program is capable of managing operating room operations by offering a comprehensive modular process which is deployed on the system platform various platform functions described herein.

The scheduling module 220 may schedule patient cases and consolidates all team members who are assigned to that procedure for communication purposes using the messaging feature embedded on a live preference card. Records, including time out and surgical counts will be collected and reported. Further, individual schedules, facility assignments, shift schedules, and call schedules are collected.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for providing a surgical workflow tool, the system comprising:
at least one user computing device in operable connection with a user network;
an application server in operable communication with the user network, the application server configured to host an application system for providing a surgical workflow tool, the application system having a user interface module for providing access to the application system through the user computing device, the application program to assist in supply chain maintenance, scheduling, and hardware integration of the surgical process of a healthcare facility.

2. The system of claim 1, further comprising a hardware integration module to permit the integration of one or more hardware components capable of communicating with the application program.

3. The system of claim 2, wherein a communication module permits the transmission of data between the one or more hardware components and the application program.

4. The system of claim 1, further comprising a display module permits the population of data via the at least one user computing device.

5. The system of claim 4, wherein the display module assists in the establishment of a supply chain.

6. The system of claim 1, further comprising an inventory module to permit a user to view supply, instrument and equipment catalogs.

7. The system of claim 6, wherein the inventory module permits the transmission of expiration dates to the user.

8. The system of claim 1, further comprising a user module to permit the input of surgeon-specific procedures.

9. The system of claim 1, further comprising an informatics module to permit the collection of data and to perform cost analysis.

10. The system of claim 9, wherein the informatics module is capable of communicating with a 3D printing system to enable a user to 3D print an instrument, a supply, or an equipment.

11. The system of claim 1, further comprising a communication module to transmit data between a hardware component and the at least one user computing device.

12. The system of claim 1, wherein the communication module permits the transmission of informatics data, scheduling data, and enables video chat sessions.

13. A system for providing a surgical workflow tool, the system comprising:
at least one user computing device in operable connection with a user network;
an application server in operable communication with the user network, the application server configured to host an application system for providing a surgical workflow tool, the application system having a user interface module for providing access to the application system through the user computing device, the application program to assist in supply chain maintenance, scheduling, and hardware integration of the surgical process of a healthcare facility;
a hardware integration module to permit the integration of one or more hardware components;
a communication module to permit the transmission of data between the one or more hardware components and the application program;
a display module to permit the population of data via the at least one user computing device;
an inventory module to permit a user to view supply, instrument and equipment catalogs;
a user module to permit the input of surgeon-specific procedures; and
an informatics module to permit the collection of data and to perform cost analysis.

14. The system of claim 13, wherein the display module assists in the establishment of a supply chain.

15. The system of claim 13, wherein the inventory module permits the transmission of expiration dates to the user.

16. The system of claim 13, wherein the informatics module is capable of communicating with a 3D printing system to enable a user to 3D print an instrument, a supply, or an equipment.
